(19) United States
(12) Patent Application Publication
(10) Pub. No.: US 2014/0051998 A1
(43) Pub. Date: Feb. 20, 2014

(54) ULTRASOUND DIAGNOSTIC APPARATUS AND METHOD FOR DISPLAYING AN ELASTIC IMAGE

(71) Applicant: GE Medical Systems Global Technology Company, LLC, Waukesha, WI (US)

(72) Inventor: Tadashi Shimazaki, Toyko (JP)

(73) Assignee: GE Medical Systems Global Technology Company, LLC, Waukesha, WI (US)

(21) Appl. No.: 13/971,121

(22) Filed: Aug. 20, 2013

(30) Foreign Application Priority Data
Aug. 20, 2012 (JP) 2012-181375

(51) Int. Cl. A61B 8/08

(52) U.S. Cl. CPC A61B 8/485
USPC 600/438

(57) ABSTRACT

An ultrasound diagnostic apparatus is provided. The ultrasound diagnostic apparatus includes a physical quantity calculating unit configured to calculate a physical quantity related to elasticity of biological tissue of a subject based on echo signals obtained by transmission/reception of ultrasound, an elastic image data generating unit configured to generate elastic image data having information indicative of a display form corresponding to the physical quantity calculated, a display unit configured to display an elastic image having the display form corresponding to the calculated physical quantity, and a calculating unit configured to calculate values related to a cardiac pulsation of the subject, wherein the calculated physical quantity and the information indicative of the display form correspond to each other such that the information indicative of the display form changes according to the calculated physical quantity over a range of physical quantities set according to the cardiac pulsation values.

### CROSS REFERENCE TO RELATED APPLICATIONS

This application claims the benefit of Japanese Patent Application No. 2012-181375 filed Aug. 20, 2012, which is hereby incorporated by reference in its entirety.

### BACKGROUND OF THE INVENTION

The present invention relates to an ultrasound diagnostic apparatus which displays an elastic image indicative of the hardness or softness of a biological tissue in a subject, and a control program thereof.

An ultrasound diagnostic apparatus which combines a normal B-mode image and an elastic image indicative of the hardness or softness of a biological tissue in a subject together and displays the result of combination, has been disclosed in, for example, Japanese Unexamined Patent Publication No. 2007-282932. The elastic image is generated in the following manner, for example. First, the transmission/reception of ultrasound is performed while deforming the biological tissue of the subject, for example. Then, a physical quantity related to the elasticity of the subject is calculated based on echo signals obtained by the transmission/reception. The physical quantity is strain, for example. Next, elastic image data having information indicative of a color corresponding to the elasticity is generated based on the calculated physical quantity. The elastic image data is generated based on information in which a physical quantity and information indicative of colors correspond to each other. In the correspondence information, the information indicative of the colors changes depending on the physical quantity in a prescribed range of physical quantity. An elastic image having a color corresponding to the elasticity is displayed based on the elastic image data generated based on such correspondence information.

Meanwhile, there has recently been a demand for evaluation of a liver disease by an ultrasound diagnostic apparatus capable of displaying an elastic image. The elastic image of the liver is generated utilizing the fact that the liver is deformed by repeating pressure to the liver due to the cardiac pulsation and its relaxation. Here, the degrees of the pressure to the liver due to the cardiac pulsation and its relaxation may differ depending on the subject. The livers having the same elasticity may differ in strain. Thus, there is a potential for elastic images to be displayed in different colors even in the case of the livers having the same elasticity.

In view of the foregoing, it is desirable that an elastic image generated in consideration of the degrees of the pressure and the relaxation be displayed.

### BRIEF DESCRIPTION OF THE INVENTION

In one aspect, an ultrasound diagnostic apparatus is provided. The ultrasound diagnostic apparatus is equipped with a physical quantity calculating unit which calculates a physical quantity related to elasticity of each part in a biological tissue of a subject, based on echo signals obtained by transmission/reception of ultrasound to and from the biological tissue, an elastic image data generating unit which generates elastic image data having information indicative of a display form corresponding to the physical quantity calculated by the physical quantity calculating unit, a display unit which displays an elastic image having the display form corresponding to the physical quantity, based on the elastic image data, and a calculating unit which calculates values related to the cardiac pulsation of the subject. In a range of physical quantities being information in which the physical quantity and information indicative of the display form correspond to each other, and being set according to the values related to the cardiac pulsation of the subject, the elastic image data generating unit generates the elastic image data, based on the correspondence information in which the information indicative of the display form changes according to the physical quantity.

According to the above aspect, the elastic image data is generated based on the correspondence information in which the information indicative of the display form changes depending on the physical quantity in a prescribed range of physical quantity set based on the values related to the cardiac pulsation of the subject. It is therefore possible to display an elastic image generated in consideration of the degrees of pressure to the biological tissue due to the cardiac pulsation and its relaxation.

Further advantages will be apparent from the following description of exemplary embodiments as illustrated in the accompanying drawings.

### BRIEF DESCRIPTION OF THE DRAWINGS

FIG. 1 is a block diagram showing one example of a schematic configuration of a first embodiment of an ultrasound diagnostic apparatus.

FIG. 2 is a block diagram illustrating a configuration of an echo data processor in the ultrasound diagnostic apparatus according to the first embodiment.

FIG. 3 is a block diagram depicting a configuration of a display controller in the ultrasound diagnostic apparatus shown in FIG. 1.

FIG. 4 is a diagram showing one example of a color conversion table.

FIG. 5 is a diagram depicting one example of a composite ultrasound image displayed on a display unit.

FIG. 6 is a block diagram illustrating a configuration of a controller in the ultrasound diagnostic apparatus according to the first embodiment.

FIG. 7 is a flowchart showing one example of the operation of the ultrasound diagnostic apparatus according to the first embodiment.

FIG. 8 is a diagram for describing a color conversion table set according to values related to cardiac pulsation.

FIG. 9 is a block diagram showing a configuration of an echo data processor in an ultrasound diagnostic apparatus according to a modification of the first embodiment.

FIG. 10 is a block diagram illustrating a configuration of a controller in an ultrasound diagnostic apparatus according to a second embodiment.

FIG. 11 is a flowchart showing one example of the operation of the ultrasound diagnostic apparatus according to the second embodiment.

### DETAILED DESCRIPTION OF THE INVENTION

Exemplary embodiments will hereinafter be described based on the accompanying drawings.

<First Embodiment>

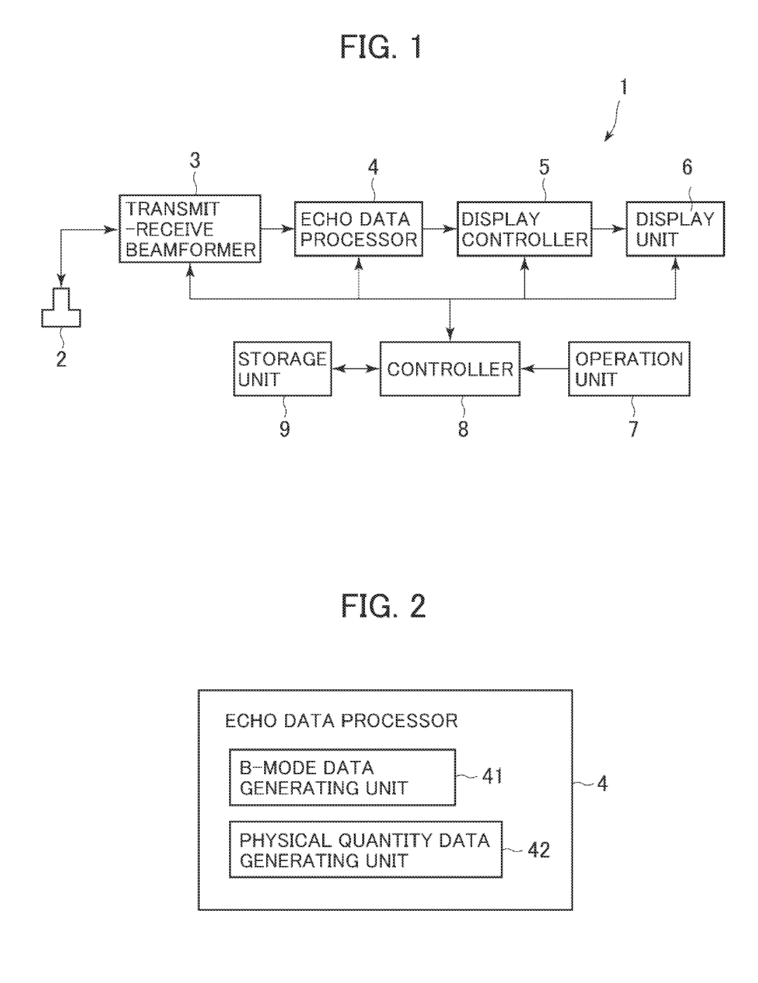

A first embodiment will first be explained based on FIGS. 1 through 8. An ultrasound diagnostic apparatus 1 shown in FIG. 1 is equipped with an ultrasound probe 2, a transmit-receive beamformer 3, an echo data processor 4, a display controller 5, a display unit 6, an operation unit 7, a controller 8 and a storage unit 9.

The ultrasound probe 2 transmits ultrasound to a subject and receives its echoes. The transmit-receive beamformer 3 drives the ultrasound probe 2 under a predetermined scan condition to perform the scanning of the ultrasound every sound ray. Also, the transmit-receive beamformer 3 performs signal processing such as phasing-adding processing on each echo received by the ultrasound probe 2. Echo data subjected to the signal processing by the transmit-receive beamformer 3 is outputted to the echo data processor 4.

As shown in FIG. 2, the echo data processor 4 has a B-mode data generating unit 41, a physical quantity data generating unit 42. The B-mode data generating unit 41 performs B-mode processing such as logarithmic compression processing, envelop detection processing or the like on the echo data outputted from the transmit-receive beamformer 3 to generate B-mode data. The B-mode data may be stored in the storage unit 9.

The physical quantity data generating unit 42 calculates a physical quantity related to the elasticity of each portion in the subject, based on the echo data outputted from the transmit-receive beamformer 3 to generate physical quantity data (physical quantity calculating function). As described in, for example, Japanese Unexamined Patent Publication No. 2008-126079, the physical quantity data generating unit 42 sets correlation windows to echo data different in time on the same sound ray at one scanning surface. The physical quantity data generating unit 42 performs a correlation computation between the correlation windows to calculate a physical quantity related to the elasticity for each pixel, thereby generating physical quantity data corresponding to one frame. Thus, the physical quantity data corresponding to one frame is obtained from echo data corresponding to two frames, and an elastic image is generated as will be described later.

The physical quantity data generating unit 42 calculates strain as the physical quantity related to the elasticity in the first embodiment. That is, the physical quantity data is data about the strain. In the first embodiment, as will be described later, pressure to the liver and its relaxation are performed by cardiac pulsation so that the liver is deformed, whereby strain is calculated.

The physical quantity data may be stored in the storage unit 9.

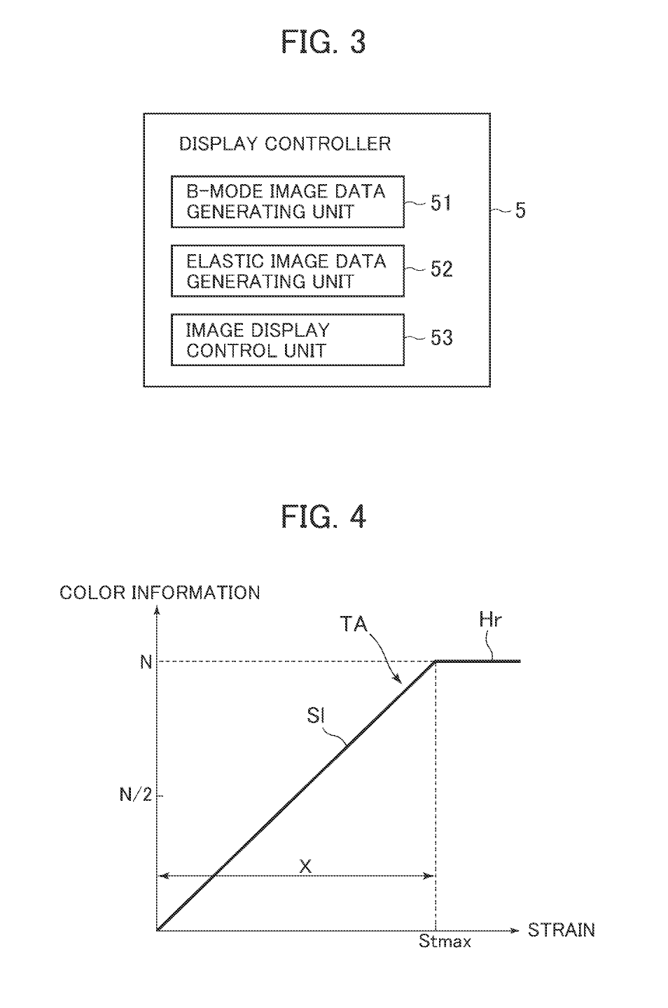

The display controller 5 is inputted with the B-mode data from the B-mode data generating unit 41 and the physical quantity data from the physical quantity data generating unit 42. As shown in FIG. 3, the display controller 5 has a B-mode image data generating unit 51, an elastic image data generating unit 52, and an image display control unit 53.

The B-mode image data generating unit 51 performs scan conversion based on a scan converter on the B-mode data to convert it to B-mode image data having information indicative of brightness corresponding to the signal intensity of each echo. The B-mode image data has information indicative of brightness of 256 levels of gray, for example.

The elastic image data generating unit 52 converts the physical quantity data to information indicative of colors and performs scan conversion based on the scan converter to generate color elastic image data having information indicative of colors corresponding to strain (color elastic image data generating function). The elastic image data generating unit 52 brings physical quantity data into gradation and generates color elastic image data includes information indicative of colors assigned to respective levels of grays. The information indicative of the display form is information indicative of each color in the first embodiment.

The elastic image data generating unit 52 converts the physical quantity data to information (hereinafter called “color information”) indicative of each color, based on a color conversion table TA to generate the color elastic image data including color information corresponding to a physical quantity.

The color conversion table TA will be explained. The color conversion table TA is information in which strain and color information correspond to each other. Color information converted by this color conversion table TA is a prescribed number of gradations (0 to N). For example, the number of gradations is 256 (N=255).

The color conversion table TA can be shown in a graph shown in FIG. 4, for example. The color conversion table TA shown in FIG. 4 takes the form of a graph having a slope part S1 and a horizontal part Hr. In the first embodiment, the range X of strain extending from zero to strain Stmax corresponds to the slope part S1.

In the slope part S1, the color information is set so as to change stepwise depending on the strain. For example, the gradation 0 is color information indicative of blue, and the gradation N is color information indicative of red. The gradation N/2 being gradation in the middle between the gradation 0 and the gradation N is color information indicative of green. In this case, the color changes from blue to green between the gradation 0 and the gradation N/2, and the color changes from green to red between the gradation N/2 and the gradation N.

The maximum value Stmax of strain in the strain range X is converted to the gradation N. Strain greater than or equal to the maximum value Stmax is converted to the gradation N. That is, strain is converted to the gradation N at the horizontal part Hr. Thus, the strain greater than or equal to the maximum value Stmax is represented in the same color (e.g., red) in an elastic image.

The strain range X is set depending on the values related to the cardiac pulsation of the subject. The details thereof will be described later. The strain range X is one example illustrative of an embodiment of a physical quantity range set depending on the values related to the cardiac pulsation of the subject.

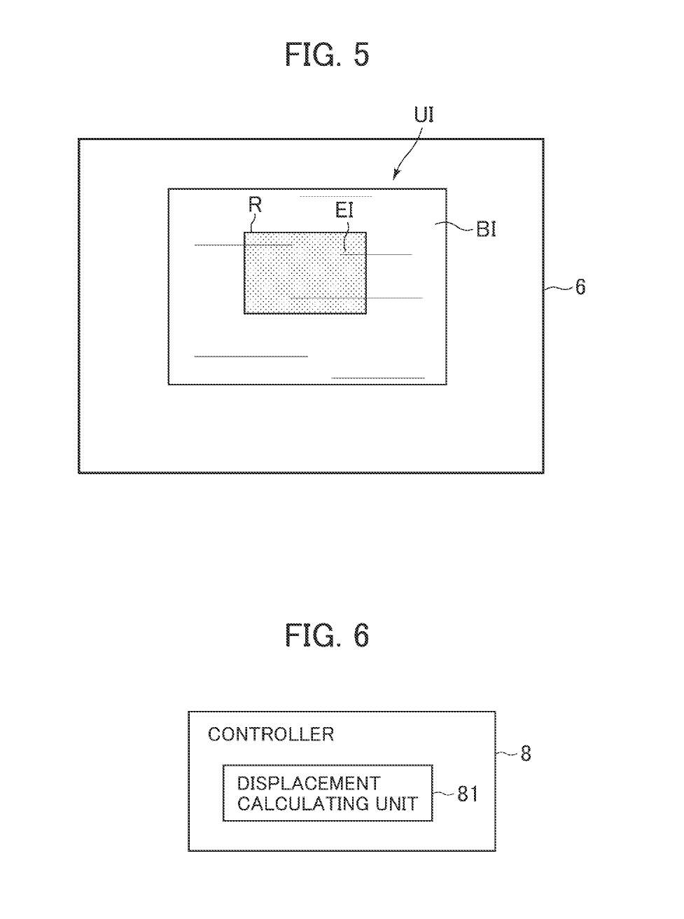

The image display control unit 53 combines the B-mode image data and the color elastic image data to generate image data of a composite ultrasound image displayed on the display unit 6. Also the image display control unit 53 causes the display unit 6 to display the image data as a composite ultrasound image UI in which a B-mode image BI and an elastic image EI are combined, as shown in FIG. 5. The elastic image EI is displayed (shown in dots) within a region R set to the B-mode image BI. The elastic image EI is an image having a color corresponding to the strain.

The B-mode image data and the color elastic image data may be stored in the storage unit 9. The image data of the composite ultrasound image may be stored in the storage unit 9.

The display unit 6 is comprised of, for example, an LCD (Liquid Crystal Display), a CRT (Cathode Ray Tube) or the like.

The operation unit 7 includes a keyboard and a pointing device (not shown) or the like for inputting instructions and information by an operator.

The controller 8 is a CPU (Central Processing Unit). The controller 8 has a displacement calculating unit 81 as shown in FIG. 6. The displacement calculating unit 81 calculates the displacement of the cardiac wall due to the cardiac pulsation (displacement calculating function). The details thereof will be described later.

The value related to the cardiac pulsation is a value measured with respect to the cardiac pulsation, such as the displacement of the cardiac wall due to the cardiac pulsation.

The controller 8 reads a control program stored in the storage unit 9 to execute the displacement calculating function. Also the controller 8 executes functions at the respective parts of the ultrasound diagnostic apparatus 1, starting with the physical quantity calculating function, the color elastic image data generating function and an image display control function in addition to the displacement calculating function.

The storage unit 9 is, for example, an HDD (Hard Disk Drive), or a semiconductor memory such as a RAM (Random Access Memory), a ROM (Read Only Memory) or the like.

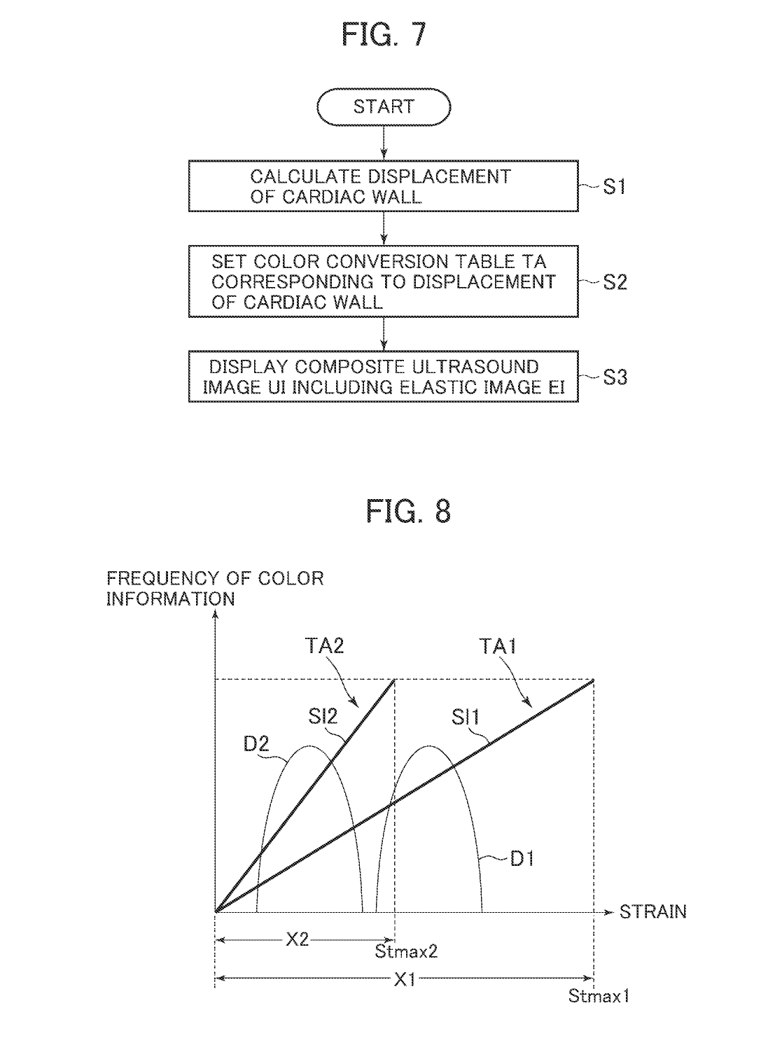

The operation of the ultrasound diagnostic apparatus 1 according to the first embodiment will now be described based on a flowchart shown in FIG. 7. A description will be made here of the operation where an elastic image EI of the liver is displayed.

First, at Step S1, the displacement of the cardiac wall is calculated. Specifically, the operator performs the transmission/reception of ultrasound on a range including the heart of the subject by the ultrasound probe 2. Then, B-mode image data is generated based on acquired echo signals, and a B-mode image including the heart is displayed on the display unit 6.

When the B-mode image is displayed, the operation sets a region of interest to the B-mode image. This region of interest is set so as to include a region that performs pressure to the liver and its relaxation at the cardiac wall.

The displacement calculating unit 81 extracts the cardiac wall in the region of interest, based on the B-mode image data. The displacement calculating unit 81 performs extract processing, based on information corresponding to the brightness of the B-mode image data. Then, the displacement calculating unit 81 performs tracking on the motion of the extracted cardiac wall, based on the B-mode image data to calculate the displacement of the cardiac wall. The calculated displacement of cardiac wall is the displacement of the cardiac wall that performs the pressure to the liver and its relaxation.

Incidentally, the operator may trace the outline of the cardiac wall in the B-mode image using a track ball or the like of the operation unit 7 without setting the region of interest to the B-mode image. A region to be traced may be only a region that performs the pressure to the liver and its relaxation at the cardiac wall. Thus, when the cardiac wall is traced, the displacement calculating unit 81 tracks the motion of the traced region, based on the B-mode image data to calculate the displacement of the cardiac wall.

When the displacement is calculated at Step S1, the elastic image data generating unit 52 sets the color conversion table TA at Step S2. Specifically, the color conversion table TA is set in which the range X of strain set depending on the displacement of the cardiac wall calculated at Step S1 is of the slope part S1 (refer to FIG. 4).

The range X of the strain is set in such a manner that the maximum value Stmax becomes large as the displacement of the cardiac wall increases, whereas the maximum value Stmax becomes small as the displacement of the cardiac wall decreases. This will be described in detail. Since the degrees of pressure to the liver due to the cardiac pulsation and its relaxation become large as the displacement of the cardiac wall increases, the deformation of the liver becomes larger. Thus, a strain distribution D1 of the liver in this case becomes a distribution including a range in which strain is relatively large, as shown in FIG. 8, for example. On the other hand, since the degrees of the pressure to the liver due to the cardiac pulsation and its relaxation become small as the displacement of the cardiac wall decreases, the deformation of the liver becomes smaller. Thus, a strain distribution D2 of the liver in this case becomes a distribution including a range in which strain is relatively small, as shown in FIG. 8, for example.

Incidentally, the strain distribution D1 and the strain distribution D2 are strain distributions of the livers having the same elasticity.

In the case of the strain distribution D1, i.e., where the displacement of the cardiac wall is relatively large, a color conversion table TA1 (in which only a slope part S11 is illustrated) is set in which a range X1 of strain is the slope part S11. The strain range X1 is a range from 0 to the maximum value Stmax1. On the other hand, in the case of the strain distribution D2, i.e., where the displacement of the cardiac wall is relatively small, a color conversion table TA2 (in which only a slope part S12 is illustrated) is set in which a range X2 of strain is the slope part S12. The strain range X2 is a range from 0 to the maximum value Stmax2. Stmax1>Stmax2, and the strain range X1 assumes a range including large strain as compared with the strain range X2. The color conversion tables TA1 and TA2 shown in FIG. 8 are however shown by way of example.

The strain range X set depending on the displacement of the cardiac wall is set in such a manner that the elastic image EI is displayed without the regions having the same elasticity being much different in color, regardless of the magnitudes of the degrees of the pressure to the liver due to the cardiac pulsation and its relaxation.

When the color conversion table TA is set at Step S2, a composite ultrasound image UI including the elastic image EI is displayed at Step S3. Specifically, the operator performs transmission/reception of ultrasound to and from the range including the liver of the subject by the ultrasound probe 2. The transmission/reception of ultrasound for generating a B-mode image and the transmission/reception of ultrasound for generating an elastic image may alternately be performed.

Here, the liver is repeatedly deformed due to the cardiac pulsation. Based on echo signals obtained from the liver in which such deformation is repeated, a composite ultrasound image including an elastic image in which the deformation has been taken as strain is generated. Specifically, when the echo signals are acquired, the B-mode data generating unit 41 generates B-mode data and the physical quantity data generating unit 42 calculates strain to generate physical quantity data. Further, the B-mode image data generating unit 51 generates B-mode image data, based on the B-mode data. The elastic image data generating unit 52 generates color elastic image data, based on the physical quantity data, using the color conversion table TA set at Step S2. Then, as shown in FIG. 5 described above, the image display control unit 53 causes the display unit 6 to display the composite ultrasound image UI in which the B-mode image BI based on the B-mode image data and the elastic image EI based on the color elastic image data are combined. The composite ultrasound image UI is a real-time image.

According to the first embodiment described above, since the color conversion table TA is set according to the displacement of the cardiac wall, it is possible to display the elastic image EI generated in consideration of the degrees of the pressure to the liver due to the cardiac pulsation and its relaxation. Regardless the degrees of the pressure and the relaxation, the regions having the same elasticity can be displayed in colors free of being much different in the elastic image EI.

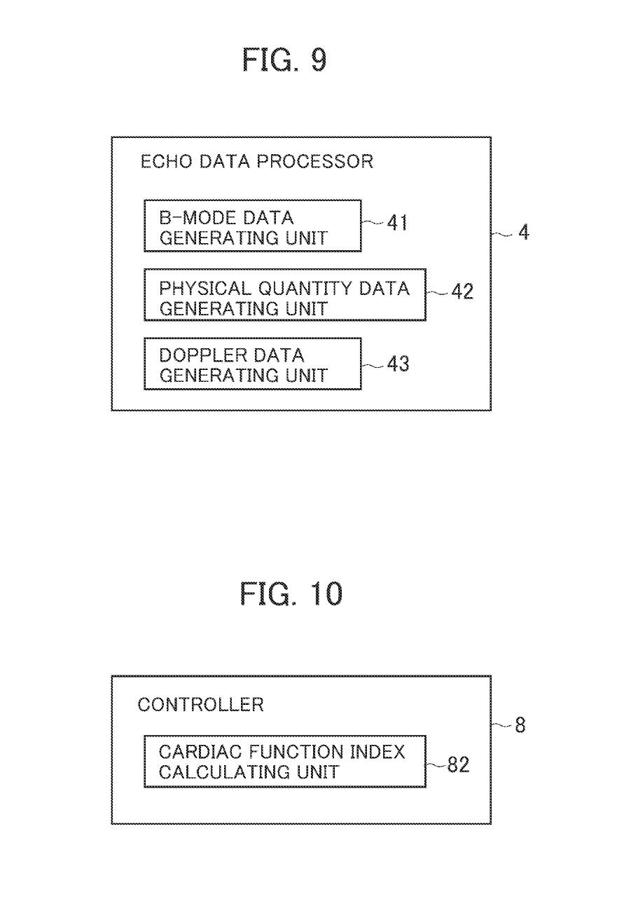

A modification of the first embodiment will next be described. In this modification, the echo data processor 4 has a Doppler data generating unit 43 in addition to the B-mode data generating unit 41 and the physical quantity data generating unit 42 as shown in FIG. 9. The Doppler data generating unit 43 performs Doppler processing including quadrature detection processing, filter processing, autocorrelation computation processing and the like on echo data outputted from the transmit-receive beamformer 3 to generate data including the velocity of a biological tissue.

The operation of this modification will be described. At the above Step 51, as described above, the operator sets a region of interest to a B-mode image in such a manner that it includes a region that performs pressure to the liver and its relaxation at a cardiac wall. The Doppler data generating unit 43 generates data including the velocity of displacement of a biological tissue in the region of interest. The biological tissue is the cardiac wall. Then, the displacement calculating unit 81 time-integrates the velocity obtained by the Doppler data generating unit 43 to calculate the displacement of the cardiac wall.

<Second Embodiment>

A second embodiment will next be described. Description of the same items as those in the first embodiment will be omitted.

In the second embodiment, as shown in FIG. 10, the controller 8 has a cardiac function index calculating unit 82. The cardiac function index calculating unit 82 calculates a cardiac function index correlated with the cardiac pulsation (cardiac function index calculating function).

The cardiac function index calculating unit 82 calculates, for example, an Ejection Fraction (hereinafter called “EF”). This EF is an index that evaluates a pump function in which the points are put to the amount of blood pumped out upon the contraction of the heart and its efficiency. The EF is calculated by the following Equation 1:

EF=100×(EDV−ESV)/EDV (%) Equation 1

where EDV is a volume of left ventricle during its diastole, and ESV is a volume of left ventricle during its systole

Since the difference between the EDV and ESV becomes large when the cardiac pulsation is high, the EF is considered to be large. On the other hand, since the difference between the EDV and ESV becomes small when the cardiac pulsation is low, the EF is considered to be small. Accordingly, the EF is considered to have a correlation with the cardiac pulsation.

The EDV and ESV are calculated by extracting the outline of the left ventricle in a B-mode image, for example. The EDV and ESV may be calculated by allowing an operator to trace the outline of the left ventricle and to track the traced outline.

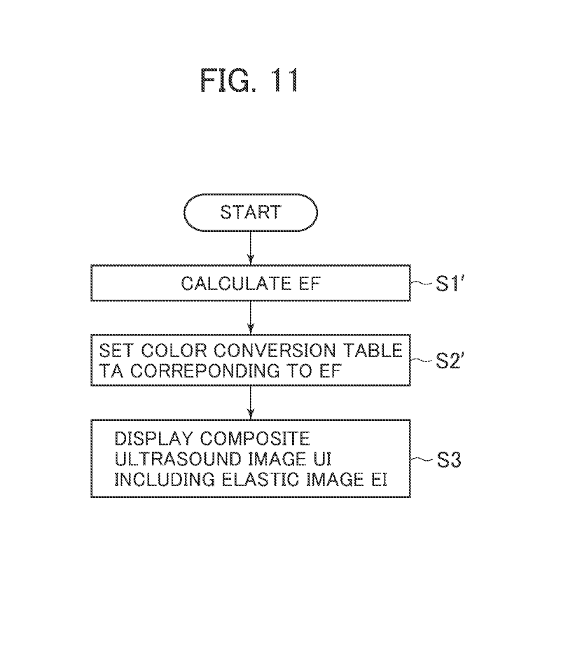

The operation of the second embodiment will next be described based on a flowchart shown in FIG. 11. The EF is first calculated at Step S1′. Specifically, as with Step S1 described in the first embodiment, the operator performs transmission/reception of ultrasound to and from a range including the heart of a subject by the ultrasound probe 2. Then, a B-mode image based on acquired echo signals is displayed.

In order to calculate the EF, there is a need to calculate the EDV and the ESV. The extraction of the outline of the left ventricle, based on B-mode image data and the tracing of the outline of the left ventricle by the operator in the B-mode image are performed to calculate these EDV and ESV. When the extraction of the outline of the left ventricle is performed, a region of interest may be set to the B-mode image by the operator. In this case, the process of extracting the outline of the left ventricle is performed within the region of interest.

The cardiac function index calculating unit 82 calculates the EDV and ESV by tracking the outline of the left ventricle, based on the B-mode image data and calculates the EF using the above Equation 1.

When the EF is calculated at Step S1′, at Step S2′, the elastic image data generating unit 52 sets a color conversion table TA in which a range X of strain set according to the EF is a slope part S1. The strain range X is set in such a manner that the maximum value Stmax increases as the EF becomes larger, and the maximum value Stmax decreases as the EF becomes smaller. This will be described in detail. Since the cardiac pulsation becomes high as described as the EF becomes larger, the deformation of the liver becomes large. Thus, the strain distribution of the liver in this case becomes a distribution marked with a symbol D1 shown in FIG. 8. A color conversion table TA1 with a strain range X1 being a slope part is set.

On the other hand, since the cardiac pulsation becomes low as described above as the EF becomes smaller, the deformation of the liver becomes small. Thus, the strain distribution of the liver in this case becomes a distribution marked with a symbol D2 shown in FIG. 8. A color conversion table TA2 with a strain range X2 being a slope part SI2 is set.

When the color conversion table TA is set at Step S2′, a composite ultrasound image UI including the liver is displayed as with the first embodiment at Step S3′.

According to the second embodiment as described above, since the color conversion table TA is set according to the EF being the cardiac function evaluation index correlated with the cardiac pulsation, there can be displayed as with the first embodiment, the elastic image EI in which the degrees of pressure to the liver due to the cardiac pulsation and its relaxation have been taken into consideration. Thus, regardless of the degrees of the pressure and the relaxation, the regions having the same elasticity can be displayed in colors free of being much different in the elastic image EI.

Although the disclosure has been explained by the exemplary embodiments as described above, it is needless to say that the methods and systems described herein can be changed in various ways within the scope of the disclosure that does not change the gist of the invention. For example, the composite ultrasound image UI is not limited to the real-time image, but may be an image based on the B-mode data and the physical quantity data stored in the storage unit 9.

Many widely different embodiments may be configured without departing from the spirit and the scope of the present invention. It should be understood that the present invention is not limited to the specific exemplary embodiments described in the specification, except as defined in the appended claims.

The disclosure is directed to an ultrasound diagnostic apparatus which displays an elastic image generated in consideration of the degrees of pressure to the biological tissue due to the cardiac pulsation and its relaxation.

1. An ultrasound diagnostic apparatus comprising:
a physical quantity calculating unit configured to calculate a physical quantity related to elasticity of each part in a biological tissue of a subject, based on echo signals obtained by transmission/reception of ultrasound to and from the biological tissue;
an elastic image data generating unit configured to generate elastic image data having information indicative of a display form corresponding to the physical quantity calculated by the physical quantity calculating unit;
a display unit configured to display an elastic image having the display form corresponding to the calculated physical quantity, based on the elastic image data; and
a calculating unit configured to calculate values related to a cardiac pulsation of the subject,
wherein the calculated physical quantity and the information indicative of the display form correspond to each other such that the information indicative of the display form changes according to the calculated physical quantity over a range of physical quantities that is set according to the values related to the cardiac pulsation of the subject, and wherein the elastic image data generating unit is configured to generate the elastic image data, based on the correspondence between the information indicative of the display form and the calculated physical quantity.

2. The ultrasound diagnostic apparatus according to claim, 1, wherein each of the values related to the cardiac pulsation is a displacement of a cardiac wall due to a heart beat.

3. The ultrasound diagnostic apparatus according to claim 1, wherein each of the values related to the cardiac pulsation is a displacement of a region that applies pressure to a liver by the cardiac pulsation at a heart.

4. The ultrasound diagnostic apparatus according to claim 2, wherein each of the values related to the cardiac pulsation is a displacement of a region that applies pressure to a liver by the cardiac pulsation at a heart.

5. The ultrasound diagnostic apparatus according to claim 2, wherein the displacement is calculated by tracking a specific region of the biological tissue in an ultrasound image of the biological tissue.

6. The ultrasound diagnostic apparatus according to claim 3, wherein the displacement is calculated by tracking a specific region of the biological tissue in an ultrasound image of the biological tissue.

7. The ultrasound diagnostic apparatus according to claim 2, wherein the displacement is calculated based on a velocity of the specific region of the biological tissue, which is calculated based on the echo signals.

8. The ultrasound diagnostic apparatus according to claim 3, wherein the displacement is calculated based on a velocity of the specific region of the biological tissue, which is calculated based on the echo signals.

9. The ultrasound diagnostic apparatus according to claim 1, wherein each of the values related to the cardiac pulsation is a cardiac function index correlated with the cardiac pulsation.

10. The ultrasound diagnostic apparatus according to claim 9, wherein the cardiac function index is an Ejection Fraction.

11. The ultrasound diagnostic apparatus according to claim 1, wherein when value related to the cardiac pulsation is a value indicative of the cardiac pulsation being relatively large, the range of physical quantities is expanded and set so as to include a physical quantity indicative of a relatively large elastic deformation.

12. The ultrasound diagnostic apparatus according to claim 2, wherein when value related to the cardiac pulsation is a value indicative of the cardiac pulsation being relatively large, the range of physical quantities is expanded and set so as to include a physical quantity indicative of a relatively large elastic deformation.

13. The ultrasound diagnostic apparatus according to claim 3, wherein when value related to the cardiac pulsation is a value indicative of the cardiac pulsation being relatively large, the range of physical quantities is expanded and set so as to include a physical quantity indicative of a relatively large elastic deformation.

14. The ultrasound diagnostic apparatus according to claim 5, wherein when value related to the cardiac pulsation is a value indicative of the cardiac pulsation being relatively large, the range of physical quantities is expanded and set so as to include a physical quantity indicative of a relatively large elastic deformation.

15. The ultrasound diagnostic apparatus according to claim 7, wherein when value related to the cardiac pulsation is a value indicative of the cardiac pulsation being relatively large, the range of physical quantities is expanded and set so as to include a physical quantity indicative of a relatively large elastic deformation.

16. The ultrasound diagnostic apparatus according to claim 9, wherein when value related to the cardiac pulsation is a value indicative of the cardiac pulsation being relatively large, the range of physical quantities is expanded and set so as to include a physical quantity indicative of a relatively large elastic deformation.

17. The ultrasound diagnostic apparatus according to claim 10, wherein when value related to the cardiac pulsation is a value indicative of the cardiac pulsation being relatively large, the range of physical quantities is expanded and set so as to include a physical quantity indicative of a relatively large elastic deformation.

18. The ultrasound diagnostic apparatus according to claim 1, wherein a maximum value of the range of physical quantities is adjusted according to the values related to the cardiac pulsation of the subject.

19. The ultrasound diagnostic apparatus according to claim 2, wherein a maximum value of the range of physical quantities is adjusted according to the values related to the cardiac pulsation of the subject.

20. A method for displaying an elastic image, the method comprising:
calculating values related to a cardiac pulsation of a subject,
calculating a physical quantity related to elasticity of each part in a biological tissue of the subject, based on echo signals obtained by transmission/reception of ultrasound to and from the biological tissue;
generating elastic image data, based on correspondence information in which the calculated physical quantity and information indicative of a display form correspond to each other such that the information indicative of the display form changes according to the calculated physical quantity over a range of physical quantities that is set according to the values related to the cardiac pulsation of the subject; and
displaying an elastic image having the display form corresponding to the calculated physical quantity, based on the elastic image data.
